(19) United States
(12) Patent Application Publication
(10) Pub. No.: US 2016/0074637 A1
(43) Pub. Date: Mar. 17, 2016

(54) Liquid collection container for a device for providing a vacuum for medical applications, and device

(71) Applicant: Paul Hartmann AG, Heidenheim (DE)

(72) Inventors: Pierre CROIZAT, Herbrechtingen (DE); Axel ECKSTEIN, Heidenheim (DE); Juana KIRSTEN, Langenau (DE)

(21) Appl. No.: 14/784,286

(22) PCT Filed: Apr. 29, 2014
(86) PCT No.: PCT/EP2014/058690

§ 371 Date: Oct. 14, 2015

(30) Foreign Application Priority Data
May 3, 2013 (DE) 10 2013 208 107.3

(51) Int. Cl. A61M 27/00

(52) U.S. Cl. CPC A61M 27/00

(57) ABSTRACT

A liquid collection container (8) for a device (2) for providing a vacuum for medical applications and for vacuum treatment of wounds in the human or animal body. The device (2) has a first housing part (4) having a vacuum-generating apparatus and the container (8) for collecting bodily fluids. The container (8) has a grip opening (24) formed for manual gripping, wherein an inner volume of the container (8) also extends into a region (28) adjacent to a wall (30) that bounds the grip opening (24). The region (28) adjacent to a wall (30) that bounds the grip opening (24) is formed by at least one chamber (32) in the interior of the container (8), that chamber being closed off in such a manner that it does not communicate with other, in particular adjacent chambers (40, 48, 45) in the interior of the container (8).

This application is the national stage of PCT/EP2014/058690 and also claims Paris convention priority from DE 10 2013 208 107.3 filed May 3, 2013.

### BACKGROUND OF THE INVENTION

The invention relates to a liquid collection container for a device, which can be worn on the body of a user and also operated in a stationary manner, for providing a vacuum for medical applications, in particular for vacuum treatment of wounds in the human or animal body, wherein the device comprises a first housing part having a vacuum-generating apparatus and the container, which can be discarded after use, for collecting bodily fluids in the interior of the container, in particular wound secretions suctioned from a wound, wherein the container comprises a second housing part of the device, which can be manually fastened to and manually detached from the first housing part of the device and, when in the fastened state, a vacuum can be applied to the interior of the container from the vacuum-generating apparatus, and wherein a connection is provided on the container for a suction line leading to the body, such that a vacuum communication can be produced between the vacuum-generating apparatus, the container and the suction line leading to the body, wherein the container has a grip opening formed for manual gripping of the second housing part, wherein an inner volume of the container also extends into a region adjacent to the wall that bounds the grip opening and, in particular, above a wall that bounds the grip opening.

Vacuum treatment means that a region of the body or wound exposed to the ambient atmosphere is sealed pressure-tight or vacuum-tight from the environment, i.e. the atmosphere in which we live and breathe, by means to be described in more detail, wherein, within the sealed wound region, a pressure that is lower than the atmospheric pressure, therefore a vacuum relative to the atmosphere, can be applied and maintained permanently. When we speak of a vacuum in this context, we mean a pressure range that is typically between 0 and 250 mmHg (millimeters of mercury) below the surrounding atmospheric pressure. This has been shown to be beneficial to wound healing. A vacuum dressing is typically used for the vacuum-tight sealing and can comprise, for example, a pressure-tight or vacuum-tight layer of film, which is typically glued onto an unaffected region of the body surrounding the wound, so that a tight seal can be achieved.

The aforementioned grip opening on the container could be constituted as a grip recess or as a grip-through opening that penetrates through the second housing part and facilitates secure gripping of the container. Such a device for providing a vacuum is known from DE 10 2009 038 130 A1 filed by the applicant. This document already states that the grip opening can be used for gripping the container that constitutes the second housing part of the device in order to fasten the container to the first housing part and/or to detach the container from the first housing part, and furthermore can be used to manually grip the entire device when the second housing part is fastened, in order to take it from one place to another or to carry it like a handbag or to fasten or position it on the body of the user in a suitable way, in particular using fastening means specifically intended for this purpose, such as belts, buckles, or clips, etc.

It has been shown to be practical when the grip is positioned in the upper area of the container that constitutes the second housing part of the device and therefore approximately above a center of gravity of the container and preferably of the device, so that the container and/or the device can be comfortably held manually and with as little exertion as possible. In this case, the fingers of a hand extend into or through the grip opening and the palm surrounds a grip area above the grip opening.

For economical reasons, weight considerations, for manufacturing reasons, and in order to achieve a large capacity, it proves beneficial to constitute the container as hollow as possible, preferably free of solid volume regions. This saves weight and material and is suitable for injection molding using twin shell forming technology. Because the container is intended to be used for bodily fluids, the problem arises that when the device is tilted, in particular in mobile operation or when the container is being fastened or detached, the bodily fluid collected in its interior is inclined to flow in the direction of gravity. In consequence thereof, the liquid also reaches regions in the vicinity of the grip opening which it wets or becomes adhered to. In the case of transparent or at least not entirely opaque materials, this leads to unpleasant impressions, when the user grips the container manually or the container and, in particular, the region of the grip opening is visible, in particular, to third parties.

The object of this invention is to address the problems described above.

### SUMMARY OF THE INVENTION

This object is achieved inventively in a device of this type in that the region adjacent to a wall that bounds the grip opening is formed by at least one chamber in the interior of the container, the chamber being closed off in such a manner that it does not communicate with other, in particular adjacent chambers in the interior of the container.

It is therefore inventively proposed that a partitioned off compartment be provided in the interior of the container adjacent to the grip opening and preferably above the grip opening, which is not connected to the remaining inner volume of the container in which liquid is collected, that is, separate in terms of pressure and liquid motion. In this way and according to the invention, liquid that has collected in the interior of the container cannot reach the partitioned off area next to the grip opening when the container is inclined or when the position of the container or the device is altered. As a result, no contamination or wetting by the liquid is visible. The device constituted in this way gives the impression of a certain distance between parts of the device to be gripped into or round manually and the bodily fluid collected inside it. This improves the appearance and also the acceptance of the device. Moreover, transparent or semi-transparent materials can be used for the manufacture of the container, which can prove advantageous when used at a certain distance from the grip opening, to be able to visually assess the level of the liquid in the container.

As already mentioned, it is advantageous if the partitioned off area in question extends above a wall that bounds the grip opening. It proves particularly advantageous if the chamber, when the container is viewed from above resting on a level surface or when the device is viewed in the vertical direction from above, extends above an entire wall that upwardly bounds the grip opening. With this further inventive idea, it is suggested that the chamber extend in such a way that, when viewed vertically from above and projected downward, it covers the grip opening. This provides full visual protection, which hides the bodily fluid stored in the container even if transparent or partially transparent materials are used, or at least makes it appear so indistinct that no unpleasant associations arise.

In a further embodiment of the invention, it is advantageous if the chamber extends above the grip opening and beyond it towards two opposite sides, so that the optically concealing effect of the chamber that is partitioned off from the remaining interior of the container is extended further toward the sides. In particular, the chamber can also extend adjacent to a wall that laterally bounds the grip opening.

In a further embodiment of this inventive idea, it is suggested that the chamber also extend adjacent to a wall that laterally bounds the grip opening, where said chamber is bounded by a wall that bounds a filter chamber. According to this further inventive idea, an inner wall of the container bounds both the filter chamber and the closed chamber provided according to the invention.

Moreover, it is suggested that, in the interior of the chamber, a further chamber that does not communicate with other chambers in the interior of the container be provided, which is used to transfer a flowing medium, in particular a rinsing medium, to the body or toward the body, and which can be fluid-dynamically connected to the first housing part and also to a tube leading to the body. When positioning and fastening the container on the first housing part, this provides a way of establishing a fluid-dynamic connection between the first housing part and the body of the user, in particular, a poorly healing wound, without having to connect a similar further separate tube with the necessary connections to the first housing part.

It proves to be practical for the chamber forming the region that is adjacent to a wall that bounds the grip opening and/or a further chamber that does not communicate with other chambers in the interior of the container to accommodate a component to be protected from moisture, in particular, an electronic component, in particular, a transponder, chip, radio communication facility, lighting facility or the like.

It also proves advantageous and practical if the interior of the container also has chambers that are characterized in that they communicate with adjacent chambers and are bounded or partitioned off at least partially by ribs or web-like walls in the interior of the container. Such a chamber is a chamber that takes up the bodily fluids in the interior of the container, that is, a liquid or exudate collection chamber, a filter accommodating chamber or an antechamber disposed upstream of the filter to which a vacuum can also be applied.

Moreover, it proves advantageous if different types of ribs are provided that are constituted between adjacent chambers or within chambers, wherein a first type of rib extends in an essentially vertical direction, i.e. up to approximately 5° with respect to the vertical, and is at a distance of at least 5 mm from the base, and wherein a second type of rib extends obliquely with respect to the vertical, that is, tilted at least approximately 30° from the vertical. It has been shown that a combination of ribs extending in the vertical direction and ribs extending obliquely with respect thereto, can effectively prevent excessive movement of liquids and, in particular, uncontrolled sloshing of liquid in the interior of the container. However, it also proves advantageous if the ribs are at a distance of at least 5 mm from the base because, in such a case, the liquid in the interior can follow quickly when the container is inclined, which is certainly desired at least in the lower region of the container in order to prevent liquid wetting a filter usually disposed in the upper region.

It also proves advantageous if, in the interior of the container, ribs of a third type are provided, which form a closed wall in a circumferential direction, that is, which join up to themselves in the circumferential direction. Such ribs can then form closed walls to bound a filter accommodating chamber or a chamber for conveying media to the body. It is also conceivable for multiple ribs of this third type to be disposed concentrically with respect to one another, wherein splash protection against sloshing liquid, in particular, for the protection of a filter, can be achieved.

It is also advantageous if ribs are provided to form chambers that communicate with one another, the ribs having openings, which preferably account for only approximately maximum 15% of their wall surface or have a maximum diameter of 4 mm, in particular, no more than 3 mm. If also proves advantageous if the ribs that extend straight or obliquely upward to an inclined or horizontal wall form an opening where they are adjacent to the wall. In such a case, air exchange can take place between chambers delimited from one another in the upward direction by ribs, which counteracts the formation of bubbles and bubbling sounds inside the container.

It also proves advantageous if the container has two mutually adjacent shells, wherein a multiplicity of ribs bounding, in particular, chambers extend perpendicularly to the dividing plane of the shells. In such a case, openings can also be formed easily in the manufacturing process in such a way that they are made up of open-edge recesses in the ribs toward the dividing plane. It proves advantageous if multiple containers with different liquid collection volumes are provided or kept. Of these containers, the shell part facing the first housing part is advantageously always identical and the other shell part is constituted with differing collection volumes, in particular, with different dimensions in the direction perpendicular to the dividing plane.

Furthermore, the object of the invention is a device with the characteristics of the independent device claim.

Further characteristics, details, and advantages of the invention result from the attached claims and from the drawings and the following description of a preferred embodiment of the invention.

The drawing shows:

### BRIEF DESCRIPTION OF THE DRAWING

FIGS. 1a, b a perspective view of a device for providing a vacuum for medical applications with a container for collecting bodily fluids which can be discarded after use;

FIG. 2 a perspective view of a half-shell of the container according to FIG. 1, viewed from the inside;

FIG. 3 a perspective view of the half-shell according to FIG. 2, viewed from the outside;

FIG. 4a the half-shell according to FIG. 3 with overmolded sealing elements;

FIGS. 4b, c two views of the overmolded sealing elements;

FIG. 5 the other half-shell of the container according to FIG. 1, viewed from the inside;

FIG. 6 the half-shell according to FIG. 5, viewed from the outside; and

FIGS. 7a, b sectional views of the container.

### DESCRIPTION OF THE PREFERRED EMBODIMENT

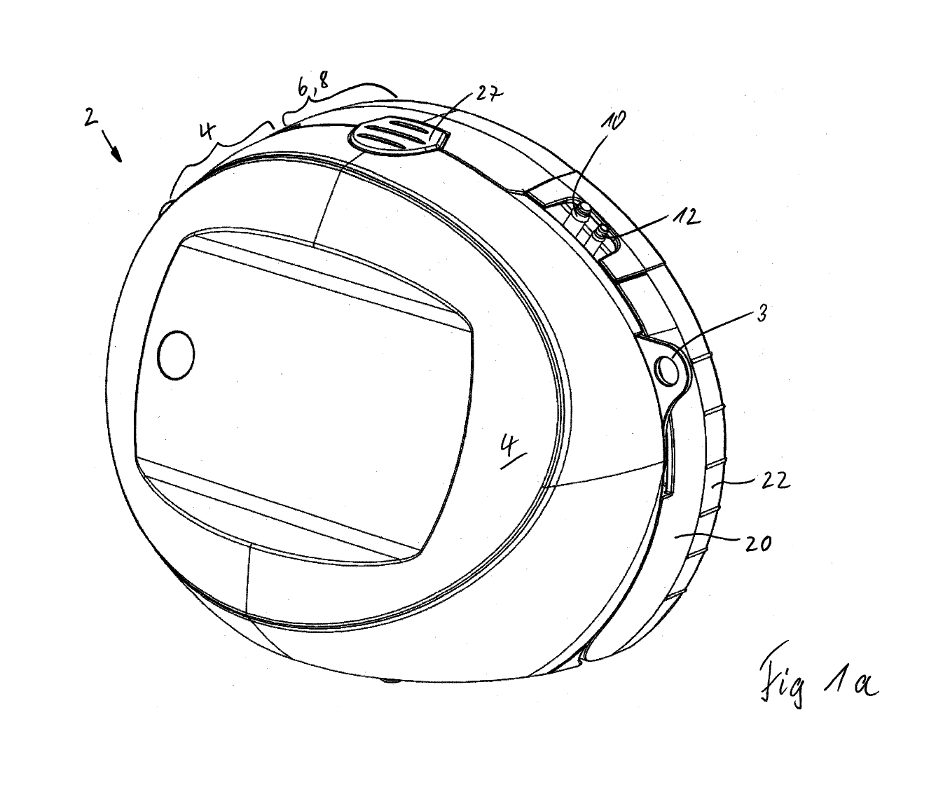

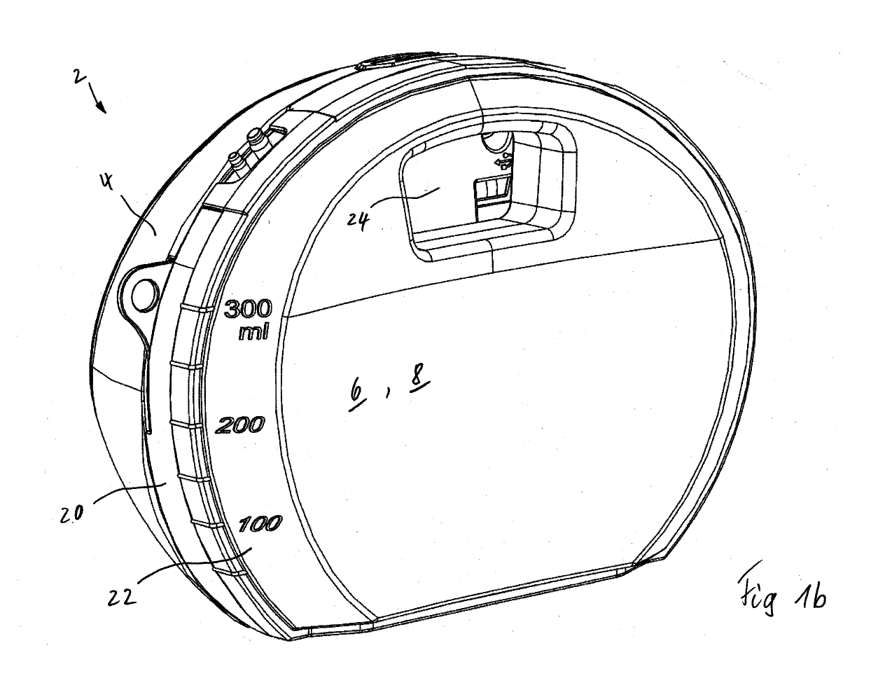

FIG. 1a, b shows a device, collectively designated with reference number 2, for providing a vacuum for medical applications. The device 2 can be worn on the body of a user (mobile operation) but can also be operated in a stationary manner (stationary operation). In mobile operation, the device could be carried by means of a strap or belt or the like and worn on the body, wherein, for this purpose, a fastening loop 3 is shown purely as an example. In stationary operation, the device can be placed on a preferably level surface. The device 2 comprises a first housing part 4 in which a vacuum-generating apparatus as well as electric or electronic control components for the device 2 as a whole are accommodated, including batteries or preferably rechargeable batteries. Moreover, the device 2 comprises a second housing part 6, which is constituted by a container 8 for collecting bodily fluids in its interior, in particular for collecting wound secretions suctioned out of a wound. The container 8 is constituted as an article, which can be discarded after use. The container 8 comprises upper connecting glands 10, 12 for a suction line not depicted and a line for feeding media toward the body of the patient or for forming a measuring duct. The suction line, which is not depicted, leads, in the exemplary operation of the device for the vacuum therapy of wounds, to a wound dressing that seals the wound in a pressure-tight manner and communicates at that location, for example via a port, with a wound region in order to create, maintain or vary a vacuum in the wound region and to suction wound exudates into the container 8. To achieve this, the container 8 communicates with the vacuum-generating apparatus in the first housing part 4.

The connecting gland 12 also leads (via a tube not depicted, which constitutes a measuring or rinsing duct) toward the body of the user, in particular, to a wound. The connecting gland 12 communicates, in a way to be described in more detail, through the interior of the container 8 with the first housing part 4, where a supplied medium, in particular a rinsing medium, can be added to it, or where it is connected to a measuring connection.

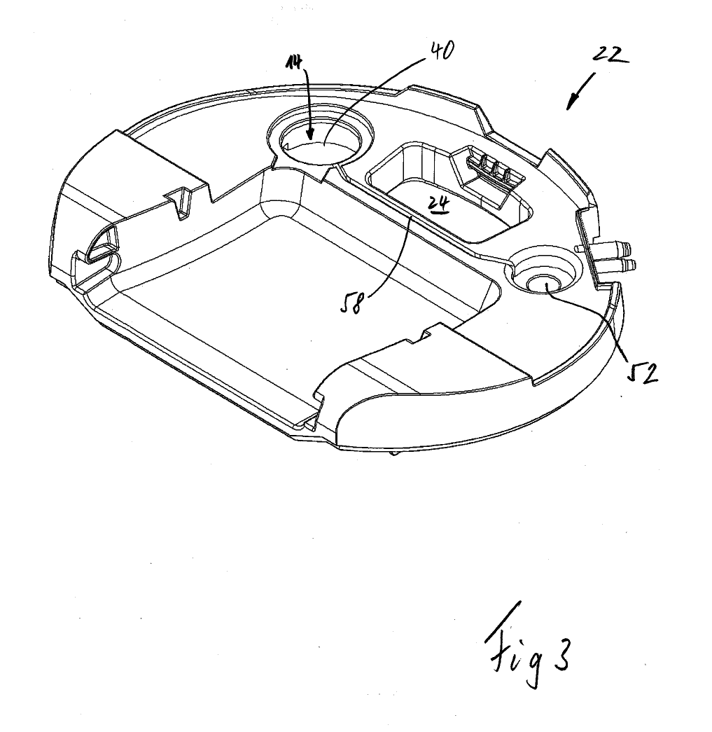

The connecting gland 10 for the suction line opens out into the interior of the container 8. The interior of the container 8 communicates via a vacuum interface 14 (FIG. 3, 4a), to be described in more detail, with the vacuum-generating apparatus within the first housing part 4 when the container 8 is fastened to the first housing part in the operating position (see in FIG. 1). For this purpose, the container 8 can be positioned manually on the first housing part 4 and then brought manually into a mechanically fastened and mechanically detachable mounting position, in particular by latching. In this mounting position, vacuum communication between the interior of the container 8 and the vacuum-generating apparatus and fluid-dynamic communication between the connecting gland 12 and the assigned measuring or rinsing connection on the first housing part 4 is also automatically achieved, in the manner basically described in DE 10 2009 038 130.9, the entire disclosure of which is hereby incorporated by reference.

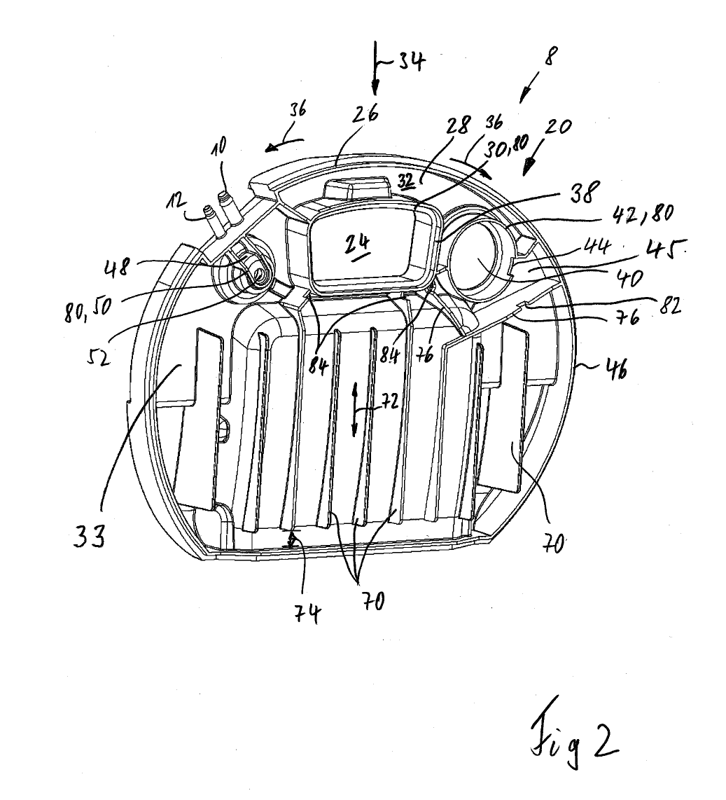

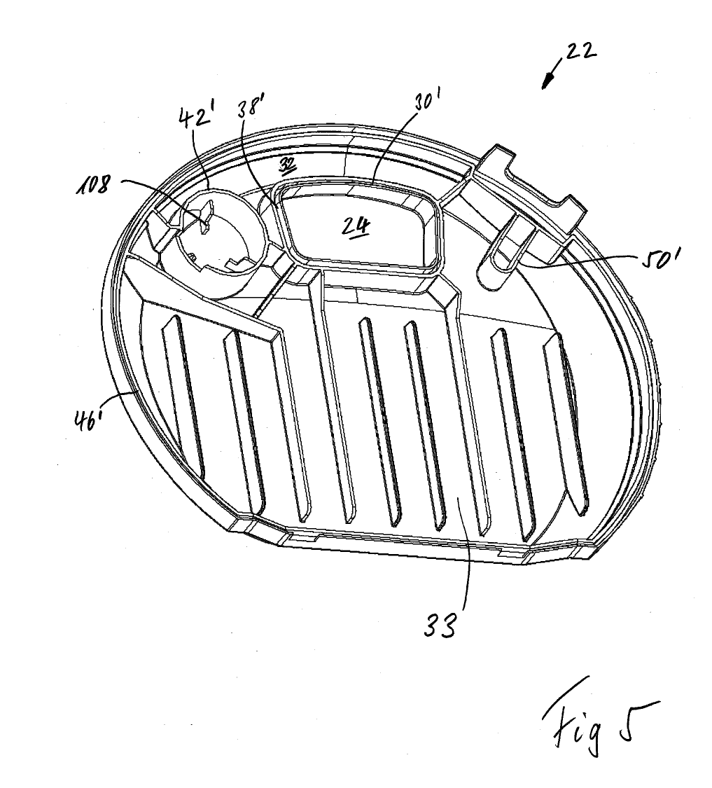

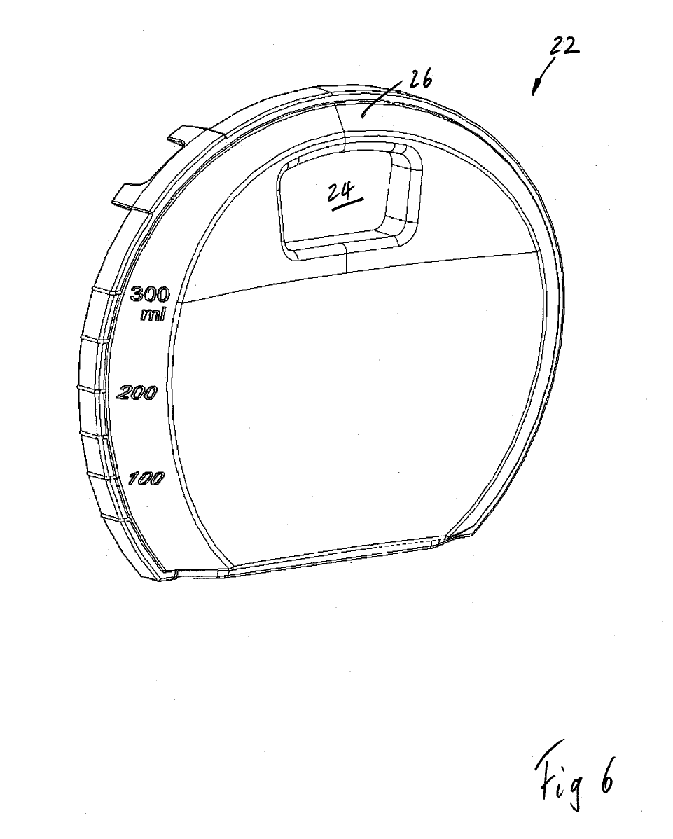

FIGS. 2 to 6 show the embodiment of the container 8 that forms the second housing part 6. The container 8 comprises a first half-shell part 20 that faces the first housing part 4 and a second half-shell part 22 that faces away from the first housing part 4 (FIGS. 5, 6), which can be placed on the body of the user in mobile operation, and which, when assembled, bound the interior of the container 8. The container 8 further comprises a grip opening 24, which, in the illustrated example, is constituted as a grip-through opening that penetrates through the container 8 and which is formed and/or bounded by the two half-shell parts 20, 22. In the first container 8, the grip opening 24 is constituted approximately in an upper quarter and centrally, so that the center of gravity of the container and of the device as a whole is located approximately below the grip opening 24. The container 8 and the entire device can be gripped by manual gripping in the grip opening 24 with the fingers of the user. At the same time, the user grips right round a relatively wide web 26, which forms a grip area, constituted above the grip opening 24 with his or her hand. When gripping round this web 26, the user can, preferably with the thumb, operate an unlocking organ 27 (FIG. 1a), so that the container is released from its operating position depicted in FIG. 1a is at the first housing part 4 and can be lifted upward at an inclined angle. This is also described in DE 2009 038 130.9, the disclosure of which has already been incorporated by reference.

In the figures, it can be seen that the interior of the container 8 extends into a region 28 adjacent to a wall 30 that bounds the grip opening 24. In the example and illustrated preferred case, this region 28 is constituted above a wall 30 that upwardly bounds the grip opening 24. This region 28 is bounded or formed by a chamber 32, which is closed off, that is, partitioned off in such a manner that it does not communicate with other adjacent chambers or regions in the interior of the container 8. This means that a body fluid suctioned into a liquid collection chamber 33 in the interior of the container 8, in particular wound exudates suctioned from a wound, cannot penetrate into this chamber 32 even if the container 8 is tipped or briefly tilted. Thus, the chamber 32 forms a kind of visual protection in the region of the grip opening 24 and in the region of the grippable web 26, when viewed from outside the container 8. In the preferred case shown by way of example, this chamber 32 extends above the entire grip opening 24, as seen from above in the vertical direction 34. The chamber 32 also extends in the direction of the arrows 36 beyond the grip opening 24 toward two opposite sides. From FIG. 2 it can further be seen that the chamber 32 also extends adjacent to a wall 38, which laterally bounds the grip opening 24. There it is bounded toward the outside by a wall 42, which forms a chamber 40 for accommodating a filter.

Unlike chamber 32, the chamber 40, which accommodates the filter, is not fluid-dynamically closed off. It communicates with the vacuum-generating apparatus in the first housing part 4 and also with an antechamber 45 via a vacuum communicating opening 44 in the wall 42. In respect of its walls, the second half-shell part 22 is constituted perpendicular to the dividing plane in a complementary manner to the first half-shell part 20. FIG. 5 shows a wall 42′ constituted in a complementary manner to the wall 42. The wall 30, 38 that bounds the grip opening 24 and which extends through the entirety of one circumferential direction, as well as an outside wall 46 and the associated complementary walls 30′, 38′, 46′ are constituted in the separation plane in their joint region in such a way that at least one step of the joining ends is constituted there, which on the one hand has a self-centering effect and on the other hand makes it easier to seal and bond the half-shell parts 20, 22.

Starting from the vacuum-generating apparatus, a vacuum can be built up in the interior of the container 8 through the filter (not shown in FIGS. 2 to 6) and through the opening 44 in the wall 42. Because the connecting gland 10 opens out directly into the interior of the container 8, a vacuum can be applied to the wound via the suction line (not shown) and bodily fluids can also be suctioned from there into the interior of the container 8.

As seen from the outside, the further connecting gland 12 opens out into a further chamber 48 that is closed off from the remaining compartment of the container 8, which is bounded by the walls 50 and correspondingly 50′ closed in the circumferential direction of the two half-shell parts 20, 22. In the depth direction, that is, perpendicular to the separation plane, a feed-through duct 52 leads from this chamber 48 toward the first housing part 4, that is, toward the outside of the container 8.

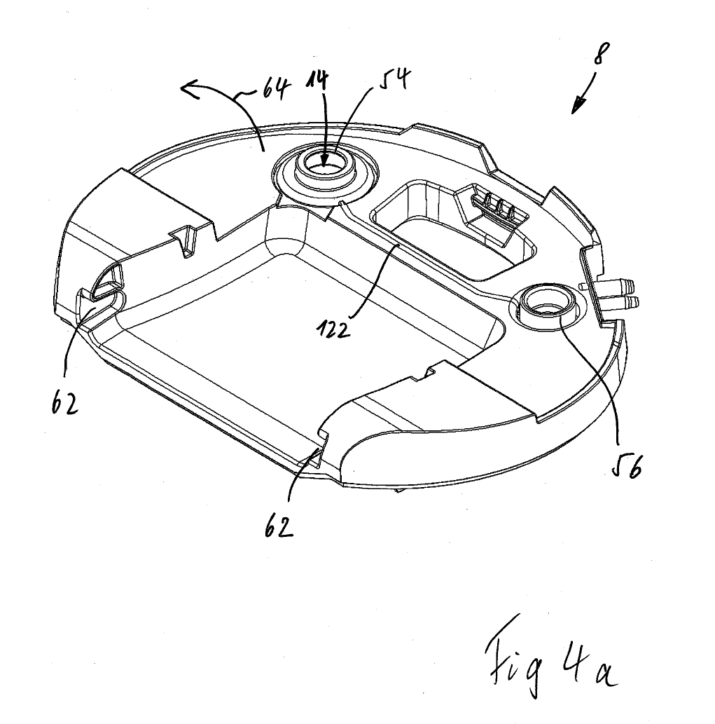

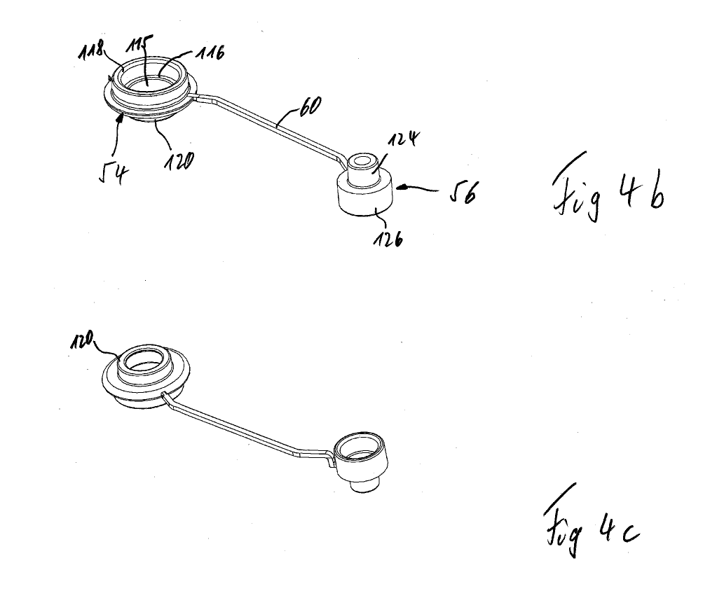

FIGS. 3 and 4 show the first half-shell part 20 from the outside, that is, looking onto the side facing the first housing part 4 in the operating position. FIGS. 3 and 4a show that a sealing element 54 and 56 can be preferably overmolded directly onto the first half-shell part 20 from the outside to seal the chamber 40 for the filter and to seal the feed-through duct 52. The narrow duct 58 is a supply channel made of injection-molded material, which forms a web 60 that connects both sealing elements 54, 56. In FIG. 4a, it can be seen that the sealing elements 54, 56 have a concentric, cylindrical section that projects outward. When the container 8 is positioned on the first housing part 4, the container 8 is placed with two bearing areas 62 that open out at the bottom roughly in a U shape obliquely from above onto a mating bearing area on the first housing part 4 constituted in a complementary manner, and then pressed in the direction of arrow 64, that is, roughly transversely with respect to the general disk shape of the container 8, against the first housing part 4, wherein the two sides latch as mentioned above. Here, the cylindrical sections of the sealing elements 54, 56 are pressed against the preferably conical connection pieces that project from the first housing part 4, wherein a sealing fluid-dynamic connection is created.

The interior of the container 8 is partitioned by a multiplicity of ribs (FIG. 2). A first type of ribs 70 essentially extend within the liquid collection chamber 33 in the vertical direction 72, that is, in the direction of the arrow 34. They terminate at the bottom at a distance 74 of preferably at least 5 mm, so that the liquid can distribute itself evenly in the interior of the container when the container 8 is tilted and during regular suction of the liquid through the connecting gland 10.

Moreover, there are ribs 76 of a second type that extend obliquely. With these ribs, sloshing of the liquid collected in container 8 toward the filter chamber 40 is prevented. Such a rib 76 bounds the previously mentioned antechamber 45 to filter chamber 40.

For the purposes of vacuum communication, a relatively small opening 82 is constituted in the obliquely extending rib 76, which establishes vacuum communication between the antechamber 45 and the remaining interior of the container 8, but which provides great resistance to penetrating liquid, which could block the filter.

Furthermore, there are ribs 80 of a third type, which join up to themselves in the circumferential direction. They form, for example, the wall 42 for the chamber 40 or the wall 50 for the chamber 48. The wall 30, which joins onto itself in the circumferential direction and which bounds the grip opening 24, can also be referred to as rib 80 of the third type.

The ribs 70, 76, which extend up as far as an inclined or horizontally extending wall also preferably have an opening 84 at the very top intended for equalizing pressure and allowing gases to pass, so that no air has to be led through the liquid.

The two half-shell parts 20, 22, are joined to each other permanently so that they form a seal preferably by gluing or thermowelding and thus constitute the second housing part 6 of the container 8 that constitutes device 2. The chamber 32, which is closed off from the liquid and the remaining interior of the container 8, protects the manually grippable web or the grip area 26 from contamination from bodily fluid collected in the interior of the container 8. It also provides visual protection, which, viewed from above, extends over the area of the grip opening 24 partially or preferably completely.

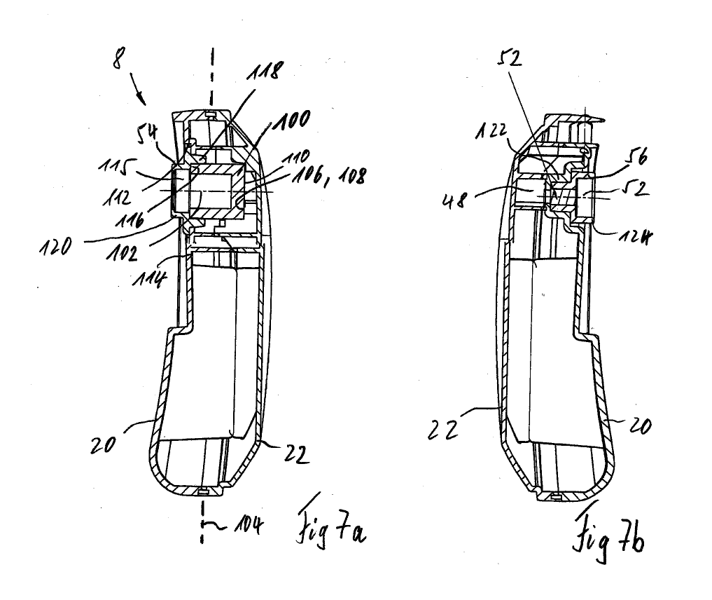

FIG. 7a shows the configuration of the filter 100 in the container 8. It can be seen that the filter 100 is disposed between the sealing element 54 and an internal area 106 of the half-shell part 22, in an axial direction 102, which also corresponds to the joint direction of the half-shell parts 20, 22 and extends perpendicularly to a dividing plane 104 between the half-shell parts 20, 22. This internal area 106 is preferably formed from preferably a plurality of ribs 108, which are particularly clearly visible in FIG. 5. They also advantageously extend in the axial direction 102 and preferably additionally radially with respect to the axial direction 102. The filter 100 can, for example, be pot-shaped, wherein the outside of the pot base 110 rests axially against the ribs 108. The sealing element 54 is made of an elastically compliant material. It is disposed in an opening 112 in a wall 114 of the first half-shell part 20 of the container 8, and preferably by being overmolded directly at that position. The sealing element 54 is preferably constituted concentrically and comprises an opening 115 and inside it a step 116, against which the filter 100 rests in the axial direction 102. This causes the sealing element 54 to deform slightly, which results in a clamping force in the axial direction 102, which holds the filter 100 in its intended mounting position. As can be seen in FIG. 7a and FIG. 4b, the sealing element 54 comprises a tubular projection 118 that projects into the interior of the container 8, into which the filter 100 engages axially. The internal diameter of the tubular projection 118 matches the outside circumference of the filter 100 in such a manner that the latter is fixed inside it by a clamping and frictional connection, which makes it easier to join together the half-shell parts 20, 24.

Moreover, the sealing element 54 comprises a tubular projection 120 that projects from the container 8 toward the first housing part 4, by means of which a fluid-dynamic connection can be created to the vacuum-generating apparatus in the first housing part 4. For example, the tubular projection 120 can be positioned against a, for example, conical projection so that it automatically seals and creates a fluid-dynamic connection when the container 8 is placed in its intended mounting position on the first housing part 4.

FIG. 7b shows a sectional view that corresponds to FIG. 7a but in the opposite viewing direction through the further sealing element 56, which is constituted as illustrated in FIGS. 4b, c. Like sealing element 54, it has tubular projections 122, 124. Its feed-through duct 52 opens into the chamber 48 mentioned above, which is sealed off from the remaining interior of the container 8 and communicates via the connecting gland 12 (FIG. 2) with a rinsing or measuring tube, which is not depicted.

Both sealing elements 54, 56, are preferably manufactured according to the same injection molding process and overmolded directly onto the first half-shell part 20. In order to have to provide no more than one gate in an injection mold to be used for this process, the two sealing elements 54, 56 are interconnected via a shared filling duct 126 and filled with elastomeric material accordingly (s. FIG. 4).

1-15. (canceled)

16. A throw away liquid collection container for a device that can be worn on the body of a user and additionally operated in a stationary manner, the device structured for providing a vacuum for medical applications or for vacuum treatment of wounds on a human or animal body, wherein the device comprises a first housing part having a vacuum-generating apparatus, the container being structured for collecting bodily fluids in an interior of the container or for collecting wound secretions suctioned out of a wound, wherein the container comprises:
structure defining a second housing part of the device, which has elements for manually fastening to and manually detaching from the first housing part of the device, wherein, in a fastened state, the container is structured to permit application of a vacuum to an interior of the container by the vacuum-generating apparatus;
a connection disposed on the container, said connection structured to communicate with a suction line leading to the body such that a vacuum communication is produced among the vacuum-generating apparatus, the container and the suction line leading to the body;
structure defining a grip opening formed for manual gripping of said second housing part, wherein an inner volume of the container also extends into a region adjacent to a wall that bounds said grip opening or above a wall that bounds said grip opening; and
structure defining at least one first chamber in the interior of the container and disposed in a region adjacent to said wall that bounds said grip opening, wherein said first chamber is closed off in such a manner as to prevent communication with other chambers in the interior of the container.

17. The container of claim 16, wherein said region adjacent to said wall that bounds said grip opening extends above said wall bounding said grip opening.

18. The container of claim 16, wherein, when the container is viewed from above resting on a level surface or when the device is viewed in a vertical direction from above, said first chamber extends above an entire wall that upwardly bounds said grip opening.

19. The container of claim 16, wherein said first chamber extends above said grip opening and beyond towards two opposite sides thereof.

20. The container of claim 16, wherein said first chamber extends adjacent to a wall that laterally bounds said grip opening.

21. The container of claim 20, wherein said first chamber extends adjacent to said wall that laterally bounds said grip opening and is bounded at that location by a wall that bounds a filter chamber.

22. The container of claim 16, wherein the interior of the container has structure defining a second chamber that does not communicate with other chambers in the interior of the container, wherein said second chamber is adapted to transfer a flowing medium or a rinsing medium to the body or toward the body, said second chamber being structured for fluid-dynamical connection to the first housing part and to a rinsing tube leading to the body.

23. The container of claim 16, wherein said first chamber and/or a third chamber that does not communicate with other chambers in the interior of the container, accommodates a component to be protected from moisture or accommodates an electronic component.

24. The container of claim 16, wherein the interior of the container also has chambers that communicate with adjacent chambers and that are bounded or partitioned off at least partially by ribs or web-like walls in the interior of the container.

25. The container of claim 24, wherein different types of ribs are constituted between adjacent chambers or within chambers, wherein a first type of rib runs in a substantially vertical direction at a distance of at least 5 mm from a base of the container and a second type of rib runs obliquely with respect to said vertical direction.

26. The container of claim 25, wherein a third type of rib forms a wall that is closed in a circumferential direction and which joins up with itself.

27. The container of claim 24, wherein said ribs have openings that account for only up to 15% of wall surfaces thereof or have a maximum diameter of no more than 4 mm.

28. The container of claim 24, wherein said ribs extend straight or obliquely upward to an inclined or horizontal wall and define an opening adjacent to said wall.

29. The container of claim 16, wherein the container comprises two mutually adjacent shells, wherein a plurality of ribs or a plurality of ribs bounding chambers extend perpendicularly to a dividing plane of said shells.

30. A device that can be worn on the body of a user and additionally operated in a stationary manner, the device structured to provide a vacuum for medical applications or for vacuum treatment of wounds on a human or animal body, the device comprising:
a first housing part having a vacuum-generating apparatus;
a container, which can be discarded after use, said container structured to collect bodily fluids in an interior thereof or for collecting wound secretions suctioned out of a wound, wherein said container comprises structure defining a second housing part, which has elements for manually fastening to and manually detaching from said first housing part, wherein, in a fastened state, said container is structured to permit application of a vacuum to an interior of said container by said vacuum-generating apparatus;
a connection disposed on said container, said connection structured to communicate with a suction line leading to the body such that a vacuum communication is produced among said vacuum-generating apparatus, said container and said suction line;
structure defining a grip opening formed for manual gripping of said second housing part, wherein an inner volume of said container also extends into a region adjacent to a wall that bounds said grip opening or above a wall that bounds said grip opening; and
structure defining at least one first chamber in said interior of said container and disposed in a region adjacent to said wall that bounds said grip opening, wherein said first chamber is closed off in such a manner as to prevent communication with other chambers in said interior of said container.
